(19) United States
(12) Patent Application Publication
(10) Pub. No.: US 2017/0112501 A1
(43) Pub. Date: Apr. 27, 2017

(54) CONDUCTIVE AND RETRIEVABLE DEVICES

(71) Applicant: The United States of America, as represented by the Secretary, Department of Health & Human Servic, Rockville, MD (US)

(72) Inventors: Bradford J. Wood, Ph.D., Potomac, MD (US); Hayet Amalou, Bethesda, MD (US)

(21) Appl. No.: 15/127,974

(22) PCT Filed: Mar. 23, 2015
(86) PCT No.: PCT/US2015/022002

§ 371 Date: Sep. 21, 2016

Related U.S. Application Data
(60) Provisional application No. 61/968,757, filed on Mar. 21, 2014.

(51) Int. Cl. A61B 17/12; A61F 2/95; A61B 18/14; A61F 2/01

(52) U.S. Cl. CPC A61B 17/12031; A61F 2/01; A61F 2/95; A61B 17/12172; A61B 17/12109; A61B 18/1492; A61F 2002/011; A61F 2002/016; A61F 2002/9528; A61F 2230/005; A61F 2230/0023; A61B 2017/00358

(57) ABSTRACT

In one embodiment, a method is disclosed in which a retrieval apparatus is coupled to a retrieval portion of an implantable device. The implantable device includes a plurality of expandable members each having a portion that comes into contact with a tissue of a subject when expanded. A force is then provided to the retrieval portion to collapse the implantable device. An electrical current is also provided to the portions of the expandable members that come into contact with the tissue of the subject via the retrieval apparatus.

### CROSS-REFERENCE TO RELATED APPLICATIONS

This application claims priority to U.S. Provisional Patent Application Ser. No. 61/968,757, filed Mar. 21, 2014. The entire content of this application is hereby incorporated by reference herein.

### GOVERNMENT SUPPORT

This invention was funded by the National Institutes of Health. The United States Government has certain rights in this invention.

### BACKGROUND

(a) Technical Field

The present invention generally relates to implantable medical devices, such as filters and stents. In particular aspects, the present invention relates to implantable devices that use an electrical current to facilitate removal of a device after implantation.

(b) Background Art

Implantable devices, such as filters and stents, typically include structures that anchor an implanted device to its surrounding tissue. For example, the inferior vena cava (IVC) is a large vein that returns deoxygenated blood to the right atrium of the heart from the lower half of the body. To prevent blood clots from reaching a subject's heart, an IVC filter may be implanted into the patient. Traditional IVC filters include hooked ends that anchor a filter to the walls of the vein thereby allowing the filter to oppose the flow of blood within the vein without moving.

While generally effective at preventing movement of a device after implantation, traditional device anchors also present challenges when attempting to remove a device from a subject. In particular, the tissue to which the device is anchored may grow around the anchors, making removal of the device increasingly more difficult over the course of time. In other words, the tumor, endothelium, mucosa, wall, etc. of the lumen artery, bronchus, IVC, bile duct, etc., may grow around the anchors or contact points of the implanted device, making retrieval of the device challenging.

Thus, there remains a need in the art for implantable devices that sufficiently anchor a device after implantation while still facilitating retrieval of the device at a later time.

### SUMMARY

As described in greater detail below, the present invention facilitates the removal of an implantable device from a subject by providing electrical current to the portions of the device that come into contact with tissue of the subject.

In one embodiment, a method is disclosed in which a retrieval apparatus is coupled to a retrieval portion of an implantable device. The implantable device includes a plurality of expandable members each having a portion that comes into contact with a tissue of a subject when expanded. A force is then provided to the retrieval portion to collapse the implantable device. An electrical current is also provided to the portions of the expandable members that come into contact with the tissue of the subject via the retrieval apparatus.

According to one aspect, the implantable device may be an inferior vena cava (IVC) filter where the portions of the expandable members that come into contact with the tissue comprise anchor members located at a distal end of the filter. In another aspect the implantable device may be a stent where the expandable members form a substantially cylindrical structure when expanded. In a further aspect, the implantable device may be an embolization basket where the expandable members are coupled at an end opposite the retrieval portion. In an additional aspect, the retrieval apparatus includes a conductive snare and the retrieval portion includes a conductive hook. In various aspects, the delivered current may be between 0.1 and 0.55 amperes and may be controllable. In some aspects, the tissue may have overgrown the portion of a particular expandable member that comes into contact with the tissue and may be a tumor. In an additional aspect, the method also includes inserting the implantable device into the subject and expanding the implantable device. In yet another aspect, the portions of the expandable members that come into contact with the tissue include hooks. In another aspect, the method also includes removing the implantable device from the subject.

In another embodiment, an implantable device is disclosed. The device includes a plurality of expandable members each having a portion that comes into contact with a tissue of a subject when expanded during implantation of the device into the subject. The device also includes a retrieval portion coupled to the plurality of elongated members configured to collapse the expandable members in response to an applied force. The retrieval portion and the portions of the expandable members that come into contact with the tissue comprise conductive material and are electrically coupled.

According to one aspect, the implantable device may be an inferior vena cava (IVC) filter where the portions of the expandable members that come into contact with the tissue comprise anchor members located at a distal end of the filter. In another aspect the implantable device may be a stent where the expandable members form a substantially cylindrical structure when expanded. In a further aspect, the implantable device may be an embolization basket where the expandable members are coupled at an end opposite the retrieval portion. In various aspects, the retrieval portion may include a hook or a screw mechanism.

In yet another embodiment, a retrieval apparatus for an implantable device is disclosed. The apparatus includes an electrical power supply and a current regulator coupled to the power supply that regulates electrical current from the power supply. The apparatus also includes a conductive snare coupled to the power supply and current regulator configured to provide a retrieval force and the electrical current from the power supply to a retrieval portion of an implantable device.

In one aspect, the power supply of the retrieval apparatus is configured to provide between 0.1 and 0.55 amperes to the conductive snare.

Additional objects and advantages of the invention will be set forth in part in the description which follows, and in part will be obvious from the description, or may be learned by practice of the invention. The objects and advantages of the invention will be realized and attained by means of the elements and combinations disclosed herein, including those pointed out in the appended claims. It is to be understood that both the foregoing general description and the following detailed description are exemplary and explanatory only and are not restrictive of the invention as claimed. The accompanying drawings, which are incorporated herein and constitute a part of this specification, illustrate several embodiments of the invention and, together with the description, serve to explain the principles of the invention.

### DEFINITIONS

To facilitate an understanding of the present invention, a number of terms and phrases are defined below.

As used herein, the singular forms “a”, “an”, and “the” include plural forms unless the context clearly dictates otherwise. Thus, for example, reference to “an antigen” includes reference to more than one antigen.

Unless specifically stated, or obvious from context, as used herein, the term “or” is understood to be inclusive.

As used herein, the terms “comprises,” “comprising,” “containing,” “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, the term “subject” is meant to refer to an animal, preferably a mammal including a non-primate (e.g., a cow, pig, horse, cat, dog, rat, mouse, etc.) and a primate (e.g., a monkey, such as a cynomolgous monkey, and a human), and more preferably a human. In a preferred embodiment, the subject is a human.

### BRIEF DESCRIPTION OF THE DRAWINGS

The above and other features of the present invention will now be described in detail with reference to certain exemplary embodiments thereof illustrated the accompanying drawings which are given herein by way of illustration only, and thus are not limitative of the present invention, and wherein:

FIGS. 1A-1B depict a retrievable inferior vena cava (IVC) filter;

FIG. 2 depicts a retrievable stent;

FIG. 3 depicts a retrievable endovascular embolization basket;

FIGS. 4A-4C depict a conductive retrieval apparatus;

FIGS. 5A-5D depict the construction of a prototype system to remove an implantable device;

FIGS. 6A-6C depict various tests performed using the prototype system; and

FIGS. 7A-7C depict the test bed and force measurement set up used for the prototype testing.

It should be understood that the appended drawings are not necessarily to scale, presenting a somewhat simplified representation of various preferred features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION OF THE INVENTION

The invention has been described in detail with reference to preferred embodiments thereof. However, it will be appreciated by those skilled in the art that changes may be made in these embodiments without departing from the principles and spirit of the invention, the scope of which is defined in the appended claims and their equivalents.

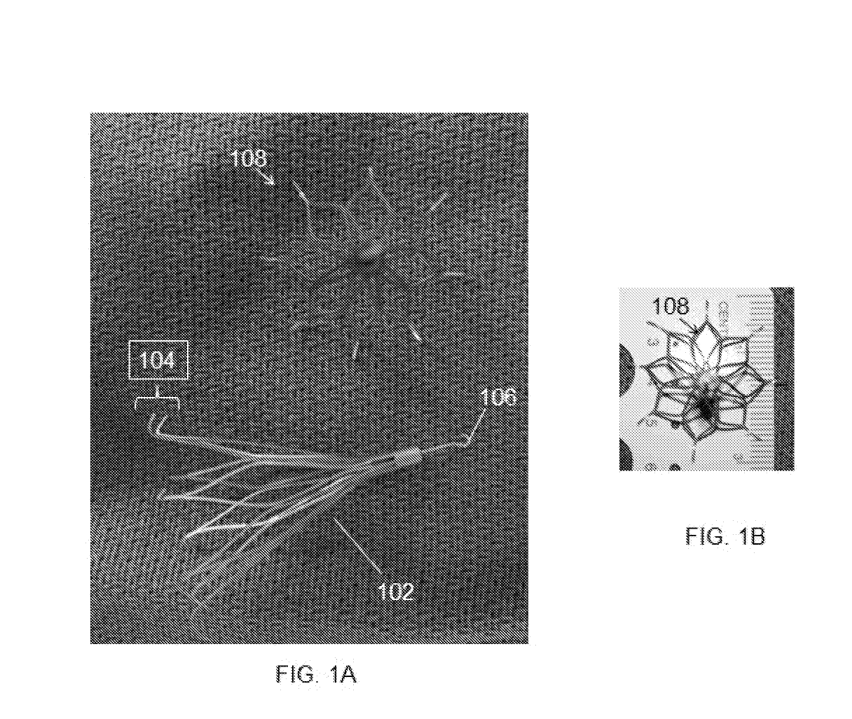

Referring now to FIGS. 1A-1B, a retrievable inferior vena cava (IVC) filter is shown, according to one embodiment. As shown, IVC filter 108 generally includes a plurality of expandable members 102 that are coupled to a retrieval portion 106. Expandable members 102 also include a plurality of anchor portions 104 at the end of filter 108 opposite retrieval portion 106. During implantation of IVC filter 108, members 102 remain in a collapsed state until positioned in a suitable location within the subject's IVC. A force is then applied to IVC filter 108, thereby causing expandable members 102 to expand within the vein and allowing anchor portions 104 to come into contact with the wall of the vein. When expanded, IVC filter 108 generally operates to trap blood clots from traveling to the subject's heart via the subject's IVC.

Retrieval of IVC filter 108 from the subject may be achieved by applying a force to retrieval portion 106 using a retrieval apparatus, thereby collapsing expandable members 102 radially inward and decouple anchor portions 104 from the wall of the vein. In various embodiments, retrieval portion 106 and the retrieval apparatus may be coupled using any suitable coupling mechanism (e.g., a hook and loop configuration, a screw mechanism, a latch mechanism, etc.).

According to various embodiments, anchor portions 104 and retrieval portion 106 are constructed using electrically conductive material and are electrically coupled to one another. During retrieval of IVC filter 108, an electric current may be applied to anchor portions 104 via retrieval portion 106 and its coupled retrieval apparatus. The amount and duration of the applied current is selected to facilitate removal of anchor portions 104 from the wall of the vein by burning through any tissue that holds anchor portions 104 to the tissue.

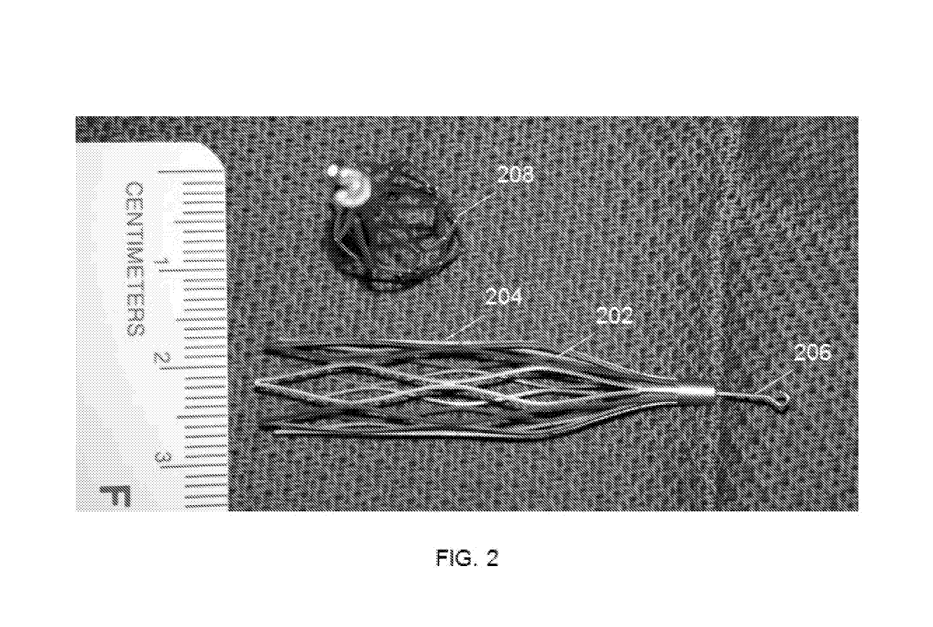

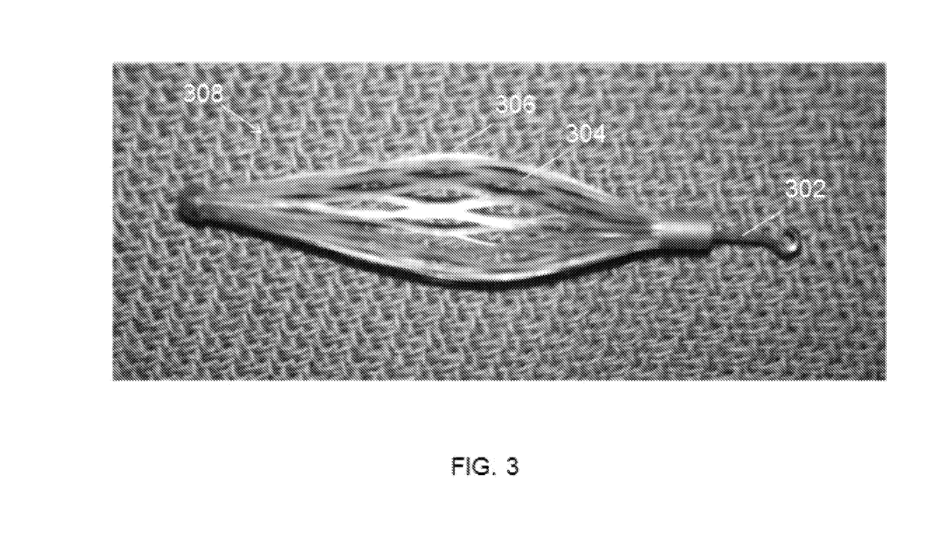

Referring now to FIG. 2, a retrievable stent is shown, according to one embodiment. As shown, retrievable stent 208 includes expandable members 202 that expand radially outward during insertion into a subject. In various cases, stent 208 may be a vascular stent, bronchial stent, or any other form of stent that provides support to a biological structure within the subject. Accordingly, stent 208 may form a generally cylindrical shape when members 202 are expanded, to allow the flow of a liquid or gas through stent 208. When expanded, portions 204 of expandable members 202 come into contact with the tissue being supported by stent 208. For example, portions 204 may come into contact with the wall of a narrow vein, to provide support to the walls of the vein and increase the flow of blood.

Extraction of stent 208 may be accomplished by a retrieval portion 206 coupled to expandable members 202. During extraction, a force is applied to expandable members 202 via retrieval portion 206 to collapse expandable members 202, thereby allowing stent 208 to be retrieved.

According to various embodiments, portions 204 and retrieval portion 206 are constructed using electrically conductive material and are electrically coupled to one another. During retrieval of stent 208 from the subject, an electric current may be applied to portions 204 in contact with the tissue of the subject via retrieval portion 206 and its coupled retrieval apparatus. Any tissue adhered to portions 204 may be burned by the applied current, thereby facilitating removal of stent 208 from the subject.

Referring now to FIG. 3, a retrievable endovascular embolization basket is depicted, according to various embodiments. Similar to IVC filter 108 and stent 208, embolization basket 308 includes expandable members 304 that expand radially outward. As shown, opposing ends of expandable members 304 are coupled to one another, allowing basket 308 to have a greater center diameter than at its opposing ends. When implanted, portions 306 of expandable members 304 come into contact with a tissue of the subject. Extraction of basket 308 may also be achieved by applying a force to a retrieval portion 302, to cause members 304 to contract. Also, as discussed in greater detail with respect to IVC filter 108 and stent 208, retrieval portion 302 and portions 306 of members 304 may be constructed using a conductive material and may be in electrical contact with one another. During extraction, a current is provided to portions 306 via retrieval portion 302, to facilitate extraction by causing portions 306 to burn through any tissue adhered to basket 308, in some embodiments.

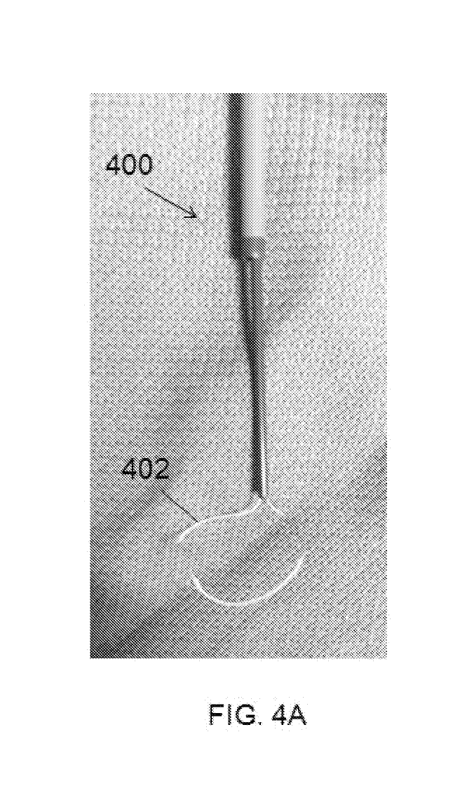

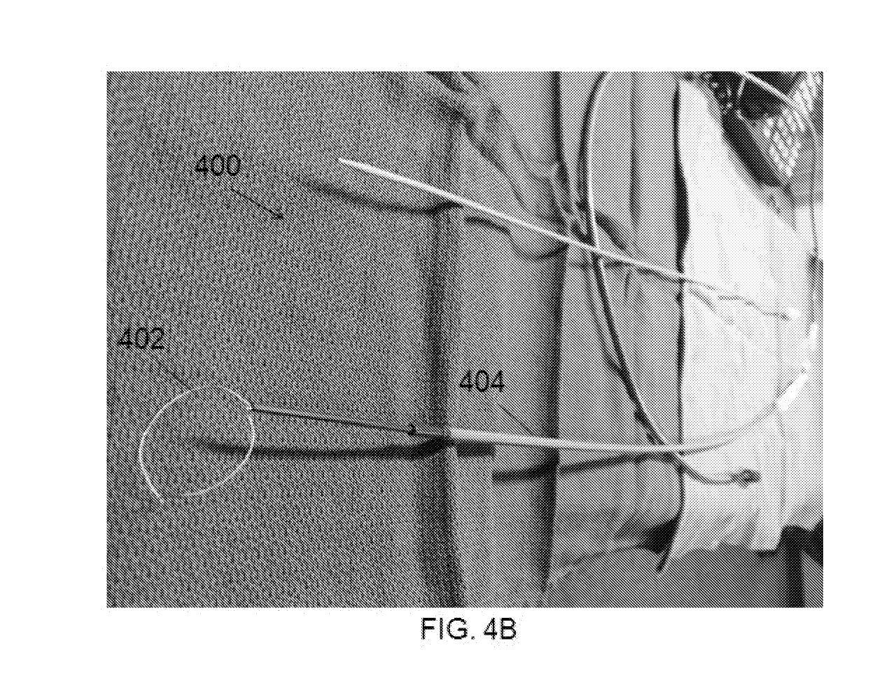

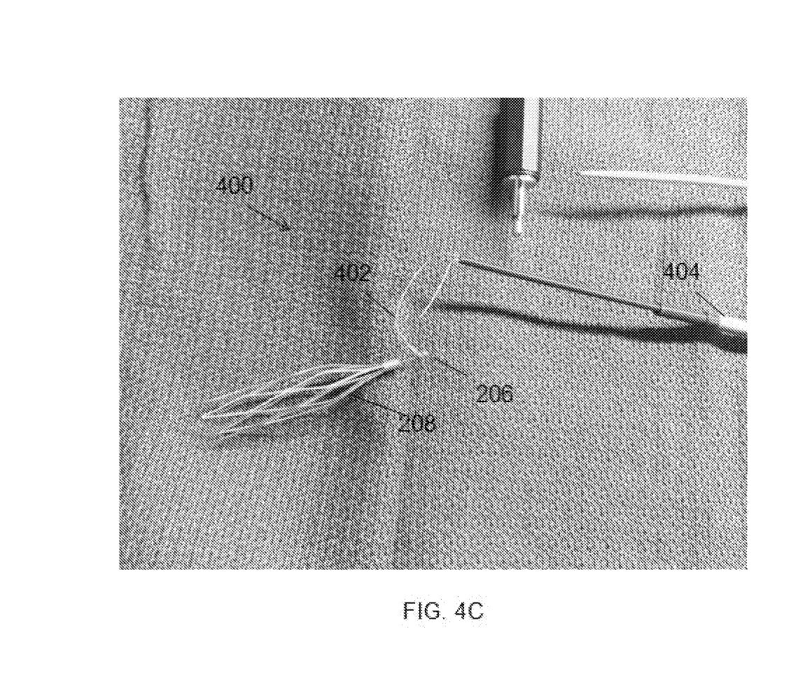

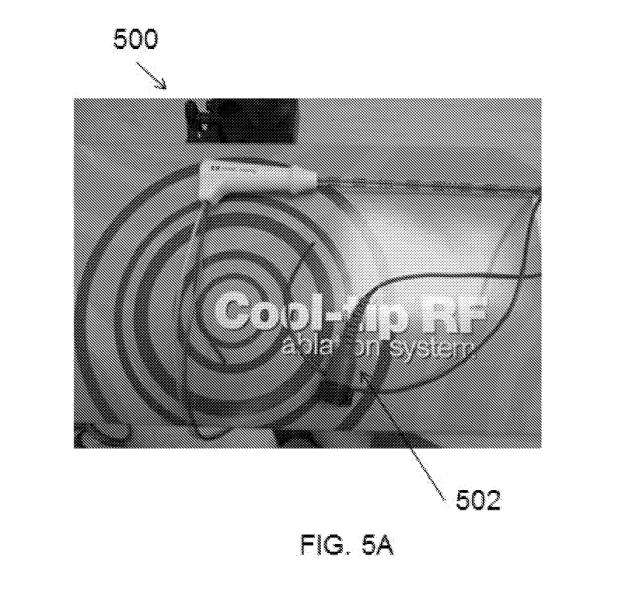

Referring now to FIGS. 4A-4C, a conductive retrieval apparatus is depicted, according to various embodiments. Retrieval apparatus 400 includes a conductive portion 402 that is configured to couple retrieval apparatus 400 to the retrieval portion of a device that has been implanted into a subject. In one embodiment, retrieval apparatus 400 may be constructed as a series of coaxial sheaths 404 having an innermost conductive element that terminates at the conductive portion 402, as shown in FIG. 4B. In other words, the conductive wire forming conductive portion 402 may be insulated from the subject (e.g., the subject's bronchial or vascular systems, etc.), except at conductive portion 402. As shown, conductive portion 402 may be of a snare configuration that can couple with any of retrieval portions 106, 206, or 302 of the implantable devices shown previously in FIGS. 1-3. For example, as shown in FIG. 4C, conductive portion 402 may be coupled to retrieval portion of 206 of stent 208 by looping the snare of retrieval apparatus 400 through the corresponding hook of stent 208. Other coupling mechanisms may also be used in other embodiments, such as a screw mechanism, a latch mechanism, or the like. As will be appreciated, a pulling force may be applied to retrieval apparatus 400 when coupled to an implanted device, to collapse the device and retrieve the device from the subject.

In various embodiments, electrical current is provided through conductive portion 402, through the coupled retrieval portion of the implanted device being removed, and into the portions of the implanted device that come into contact with tissue of the subject. For example, an extra-corporeal power supply having a current regulator may be electrically coupled to the internal conductor of retrieval apparatus 400 and conductive portion 402, thereby delivering current to the implanted device. The amount of current delivered to the implanted device may be controlled by a user through operation of the current regulator. Thus, electrical current may be used to facilitate the removal of the device by burning through any tissue adhered to the implanted device. According to some embodiments, suitable frequencies, electrical currents, and durations may be found in U.S. Pat. No. 7,122,033 entitled “Endoluminal Radiofrequency Cauterization System,” by Bradford J. Wood, the entirety of which is hereby incorporated by reference.

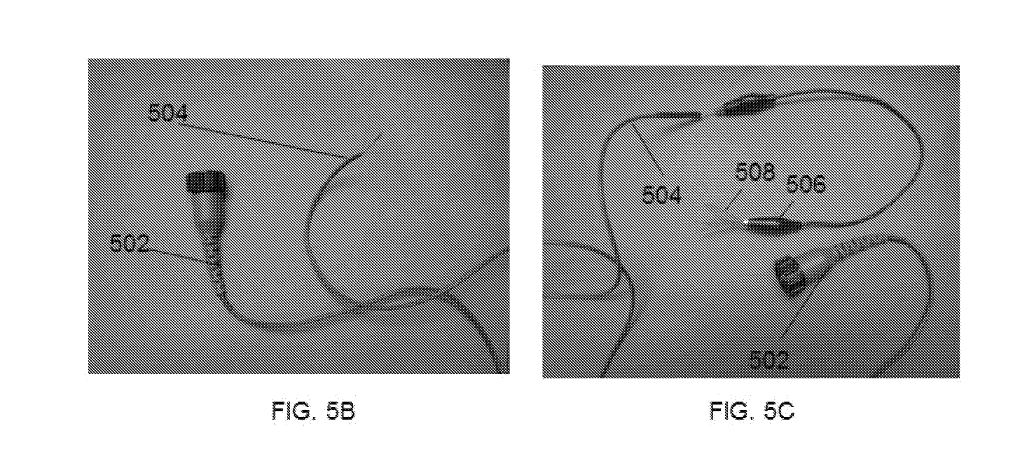

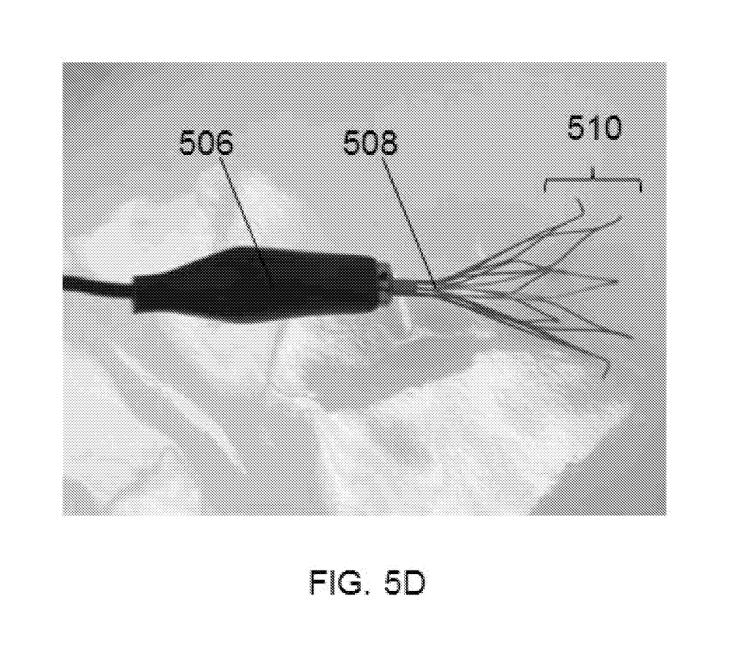

FIGS. 5A-5D depict the construction of a prototype system to remove an implantable device, according to various embodiments. In FIG. 5A, a radio frequency (RF) monopolar ablasion system 500 was modified to provide electrical current to a prototype device. In particular, a Covidien™ Cool-tip 200 Watt, 480 kHz RF monopolar ablation system was modified for purposes of prototyping the device retrieval system. As shown in FIGS. 5B-5C, an electrical cord 502 of ablation system 500 was cut at an end 504 and spliced to an alligator clip 506 that was then clamped to the apical retrieval hook (e.g., the retrieval portion) of implantable device 508. As shown in greater detail in FIG. 5D, implantable device 508 is IVC filter having anchor portions 510 configured to grip tissue of a subject when implanted. In various embodiments, anchor portions 510 and the retrieval hook of device 508 are constructed using conductive material and are electrically coupled to one another. Prototype systems were constructed in a similar manner using stent 208 and embolization basket 308 previously discussed with respect to FIGS. 2-3.

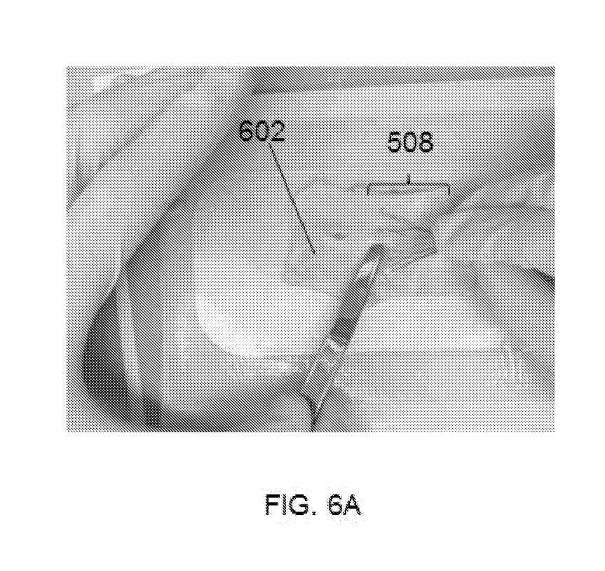

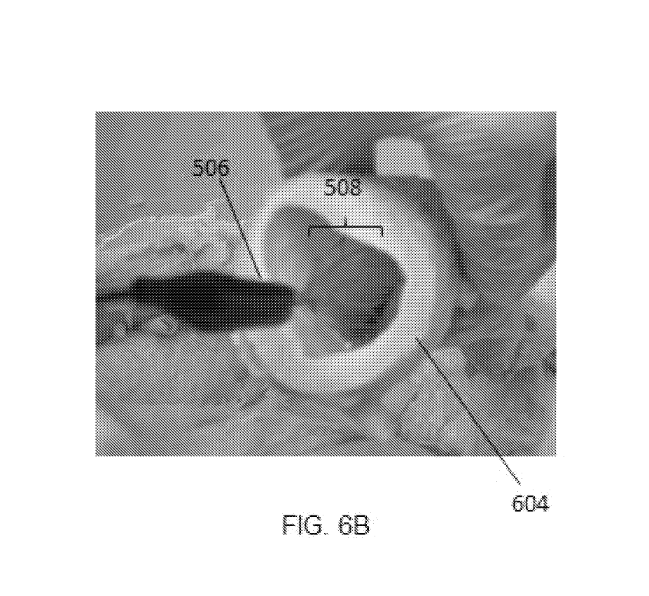

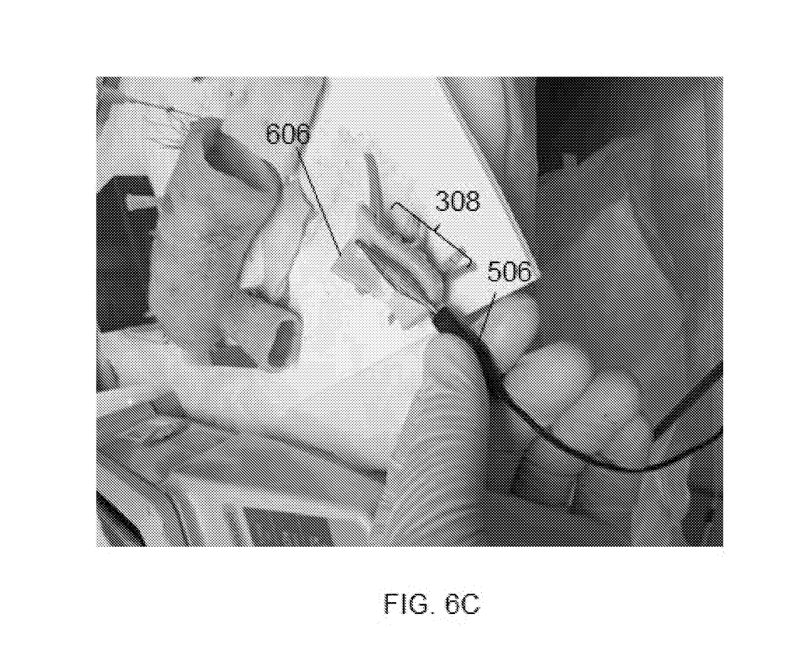

FIGS. 6A-6C depict various tests performed using the prototype system of FIGS. 5A-5D, according to various embodiments. In general, freshly harvested bovine and porcine tissues were used during testing to assess the amount of adhesion between an implantable device and the tissues after application of a current to the device. In some tests, the tissues were stretched flat on adhesive grounding pads, and two feet (e.g., anchor portions 510) of the custom fabricated device 508 were placed through the sample IVC tissue into holes left from 18G needle punctures. In FIG. 6C, embolization basket 308 was tested in a similar manner by wrapping tissue 606 around basket 308 to simulate an implanted condition.

During some tests, electrical current was applied for between 30 seconds and 3.5 minutes, or until the impedance rose at 4 different currents: 0, 0.2, 0.4, and 0.55 amps. However, since the testing was completed in vitro, it is to be appreciated that different values may be used in vivo (e.g., when less impedance is present and there is more convective heat loss due to blood flow), in various embodiments. Anchor points 510 (e.g., the points of contact between the sample tissue and the implantable device) were then removed from a scale, with the grounding pad stuck to the scale. All four group specimens were placed in saline and specimens and holes analyzed with Scanning Electron Microscopy (SEM) and H & E histology, to assess the degree and thickness of damage to the tissue. Experimental results are shown below in Table 1:

TABLE 1

| Current | Control | Low | Medium | High |
|---|---|---|---|---|
| Amps | 0 | 0.2 | 0.4 | 0.55 |
| Time (min:sec) | 0 | 0:30 sec | 3:20 | 3:55 |
| Negative weight | 4 grams | 34 grams | 107 grams | >200 grams |
| for removal | | | | |

As shown above in Table 1, varying durations and amounts of electrical current were applied, following a standard rate of removal of anchor portions 510 from the tissue. A maximum negative weight was used to estimate adhesion or the ease of retrievability of device 508. This defined a threshold for “overcooking” which resulted in charring and made device 508 more adherent. As will be appreciated, in comparison to a control in which no current was provided to device 508, the application of a current was shown to significantly improve removal of device 508 from the tissue.

Additional testing was performed using a prototype system similar to the one described above to investigate the rationale and refine the methodology of applying RF current to a custom conductive IVC filter to facilitate removal of the filter in an ex vivo porcine IVC tissue bench-top test bed.

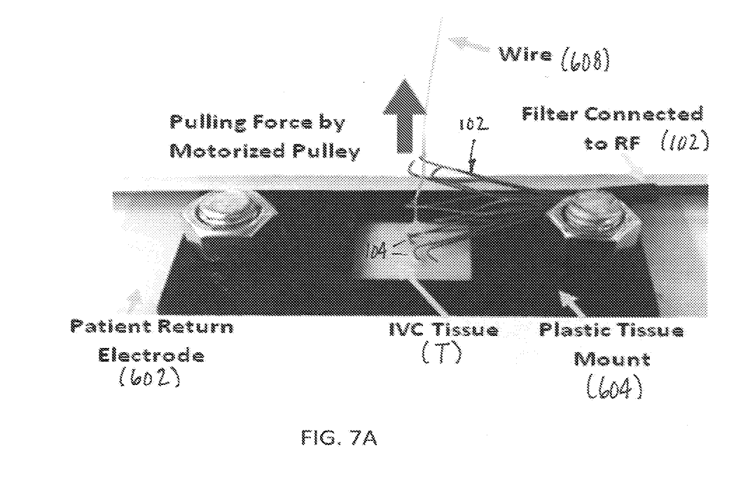

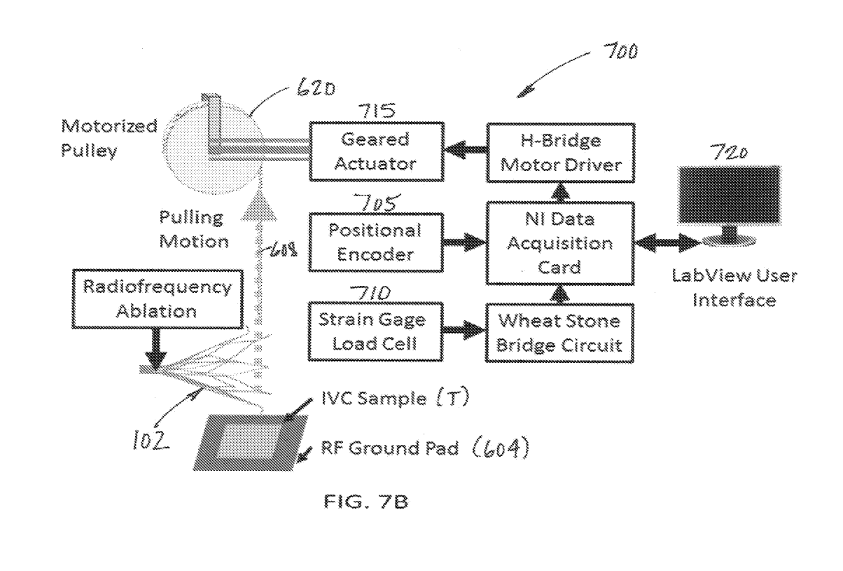

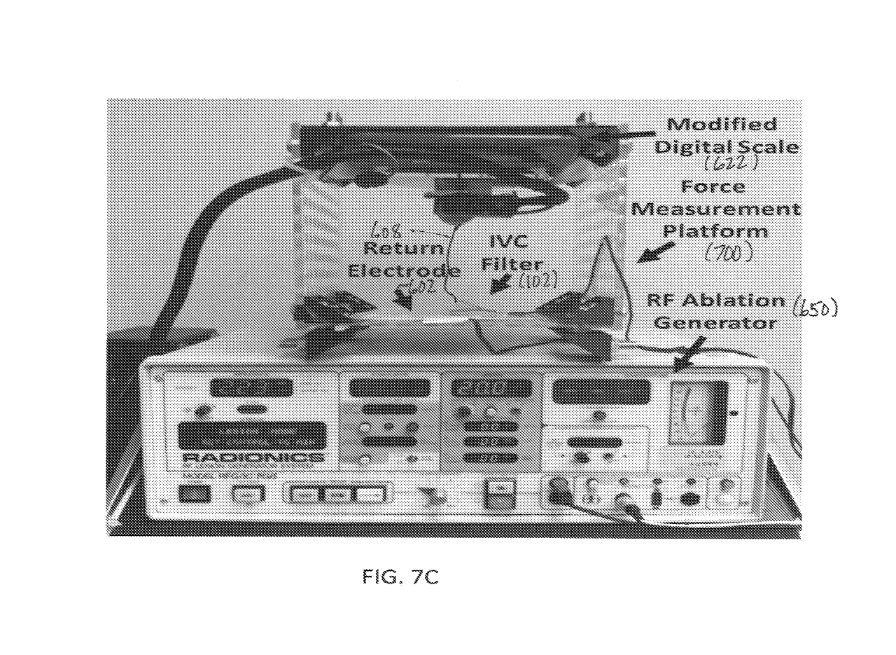

An ex vivo test bed and experiment with a custom built force measurement device was designed to determine the force required for removal of the filter after a radiofrequency current was applied to ex-vivo porcine IVC wall via conductive IVC strut legs at a specified amperage and for a designated duration. FIGS. 7A-7C depict the test bed and force measurement set up used for the testing.

Fifteen samples were tested under a variety of ablation parameters, followed by Scanning Electron Microscopy (SEM), Hematoxylin and Eosin (H & E), and Movat Pentachrome (MP) histology to study the optimal ablation setting for the removal of the filter, as well as the variable mechanical, physiological and physical implications of applying current at baseline and then at different time points.

The porcine IVC was cut longitudinally and the adventitial surface was placed face-down on the patient return electrode 602 so the luminal surface. A custom plastic tissue mount 604 was used to secure the tissue “T” on the patient return electrode 602 layered with electrode gel. Normal saline was poured on the luminal tissue to improve conductivity prior to the placement of the filter legs into the luminal side of the IVC. A custom IVC filter 102 was used to complete the ex vivo bench studies. One end of a wire 608 was tied to the distal end of the filter 102 and the other end was tied to a motorized pulley 620 of the modified digital scale 622. The anchors 104 of the two filter legs were fully submerged into the luminal surface of the tissue by consecutive manual placement of each individual filter leg.

After placement of the filter legs into the tissue, the apical retrieval hook of the filter 102 was connected to the RF lesion generator system 650. The ablation generator 650 was modified to deliver electric current to the filter 102 by custom splicing the electrical cord to connect to the apical retrieval hook of the IVC filter. The RF generator 650 was then used in lesion mode to deliver 100 mA, 200 mA, and 300 mA of current to the IVC filter for 0, 3, 5, 10, 20, and 30 seconds. After the completion of ablation, the IVC filter legs were lifted using a motorized pull wire 608 at a constant speed of 200 μm/sec.

A force measurement platform 700 (FIG. 7B) was custom designed to record the force profile during removal of the IVC filter from the tested sample. The platform consists of a positional encoder 705, a force gauge 710, an actuator 715 containing a motor and gearbox, a plastic tissue mount and acrylic sheets fabricated with a laser cutter. A graphic user 720 interface was developed that displays the force profile obtained during the experiment.

Force profile measurements were taken to determine the maximum force required to dislodge the filter legs from the wall of the vena cava, and this force is defined as the IVC filter removal force.

Successful ablations were conducted on 21 samples followed by IVC filter retrieval using the force measurement device. Samples were ablated at 100 mA, 200 mA, and 300 mA for 0, 3, 5, 10, 20, and 30 seconds. Ablations were completed at 100 mA on 15 samples, 200 mA on 3 samples, and 300 mA on 3 samples. Within the 200 mA ablation group the tissues ablated for 5 seconds had the smallest mean removal force of 96.7 grams with a standard deviation of 8.0 grams, while tissues ablated for 10 seconds had the largest mean removal force of 109.5 grams with a standard deviation of 12.8 grams. Within the 300 mA ablation group the tissues that were not exposed to electrical energy had the smallest mean removal force of 104.3 grams with a standard deviation of 11.8 grams, while tissues ablated for 20 seconds at 300 mA had the largest mean removal force of 128.0 grams with a standard deviation of 1.1 grams.

One-way ANOVA conducted on the 200 mA and 300 mA groups revealed that the differences between removal forces of tissues ablated at 0, 3, 5, 10, 20, and 30 seconds were not statistically significant with p-values >0.05. Gross observation revealed that ablation at 200 and 300 mA for 30 seconds resulted in a non-localized tissue damage that extended from the legs of the filter outwards. The ablation time of 30 seconds at 300 mA also led to tissue boiling localized to the area immediately around the filter.

The mean removal force of the control samples was 110.2 grams with the standard deviation of 24.8 grams. Samples ablated at 100 mA showed a trend that depicted an initial decrease in removal forces during the first 3 to 5 seconds followed by a return to baseline and an eventual increase after tissue ablation for 30 seconds. The tissues ablated at 100 mA, for 5 seconds had the smallest mean removal force of 64.4 grams with a standard deviation of 22.1 grams, while tissues ablated for 30 seconds at 100 mA had the largest mean removal force of 138.5 grams with a standard deviation of 36.0 grams. The absolute maximum removal force of 199.7 grams was observed after ablation of sample 13 for 30 seconds, while the absolute minimum removal force of 34.8 grams was observed after ablation of sample 10 for 5 seconds (FIG. 4). The smallest removal forces were observed after 5 seconds of ablation in 12 out of 15 trials (80.0%) and 3 seconds of ablation in 3 out of the 15 trials (20.0%). The mean removal force for tissues ablated for 3 seconds was 86.2 grams with a standard deviation of 25.3. The maximal removal force was observed at highest frequency in 12 out of 15 trials (80.0%) in tissues ablated for 30 seconds.

One-way ANOVA conducted on samples ablated at 100 mA for all time intervals revealed that differences in removal force were statistically significant between all groups with an F (5, 84)=10.69 and a p-value <0.05 (5.72×10⁻⁸).

Tissue processing and analysis was conducted on tissues ablated at 100 mA for 0, 5, and 30 seconds. Mechanical injury was observed in all samples. Tissue samples ablated at 0 and 5 seconds showed signs of local mechanical injury with cellular nuclei intact and elastic fiber disruption, while tissues ablated at 30 seconds showed transmural mechanical and thermal injury with presence of pyknotic cellular nuclei. Only the 30 seconds group showed tissue damage that consisted of thermal injury with evidence of pyknotic nuclei and signs of connective tissue denaturation, while the 5 seconds group showed absence of tissue coagulation.

The results of our ex vivo experiment show that tissue exposure of 100 mA for a shorter period of time leads to smaller retrieval forces, while larger forces are required to retrieve the filter from tissues that have been exposed to longer ablation times. The difference in the removal forces between the groups exposed to the 5 seconds and 30 seconds of ablation may be driven by a transition in tissue properties from a state in which the frictional forces are lowest between the anchor and the tissue with a smaller dose of electrical energy and increase with longer tissue exposure to electrical energy. The retrieval forces at 10 and 20 seconds compared to control are not statistically significant, which could indicate that between a smaller and higher dose of electrical energy, there is a transition zone in which the changes in tissue structure does not significantly affect the retrieval force compared to samples that have not been ablated.

The histologic change observed from H&E and MP show evidence of irreversible thermal damage from the radiofrequency in only samples exposed to ablation for 30 seconds. The absence of the histologic thermal injury in samples exposed to 5 seconds of ablation indicates that the electrical energy does not penetrate beyond the zone of mechanical injury that was caused by the removal of the IVC filter anchors by the retrieval device.

The clinical implications of the study show that when applying radiofrequency to facilitate the retrieval of the IVC filter, a transient time period exists in which the adhesive effect between the tissue and anchor decreases, allowing enough tissue disruption to ease the retrieval of the IVC filter. In this study clinically significant time periods for decreased removal force were at 3 and 5 seconds, while increased retrieval force was at 30 seconds. The higher energy levels of 200 mA and 300 mA did not show significant differences in the retrieval force between time intervals. High energy levels also led to non-localized tissue damage affecting a wide area that radiates distally from the filter strut. During the experiment, gross observations of tissue ablation and charring were present in samples exposed to 200 and 300 mA for 20 seconds and greater. After ablation and tissue coagulation there was an inability to maintain the electrical connectivity between the tissue and the electrode to continue to deliver electrical energy through the filter legs.

Although the retrieval force was lowest after ablation of tissues at 100 mA for 5 seconds, these exact energy levels may not apply directly to the clinical environment. The contribution of frictional forces in the ex vivo experimental setup are due to the interaction of the anchor with the tissue. The in vivo environment also subjects the filter to inflammatory processes that leads to the endothelialization of the individual filter anchors to the IVC wall. The required force needed to remove IVC filter anchors that have endothelialized to the wall of the vein are unknown. The endothelialization that occurs in vivo will provide additional resistance, which may require a higher deposition of energy to facilitate retrieval. Additionally, the effect of convective cooling driven by blood flow in the region of thermal ablation could lead to circumstances in which the electrical energy is not adequately deposited in the tissue site.

The current experimental setup tested a total of two struts submerged into the tissue. The in vivo setting requires all 8 struts of the IVC filter to be deployed. These 8 struts may require a larger force to retrieve due to the additive effect of the resistance of the individual struts. Additionally, the applied energy levels required to obtain a minimal force of retrieval may be different in order to dislodge 8 filter struts instead of 2.

Other embodiments will allow for application of radiofrequency monopolar energy or AC electricity to a basket or stent like device with the purpose of vessel occlusion or stopping blood flow or hemostasis, via a similar conductive element wire or snare lasso that is delivered to a hook receiver of the apex of the endovascular device. In this embodiment, the device may be left in for augmentation of vessel occlusion, or removed.

A series of test were performed on three live pig specimens which resulted in successful embolization of a pig aorta, common iliac vessels, renal arteries, and gastrointestinal arterial branches with several minutes of RF current applied to the following devices: commercial cope vascular wire, an embolization coil, a custom fabricated NIH embolization basket, snare delivery of current to basket, or other intravascular devices. Below is a table which summarizes the tests that were performed and identifies the vessel in which the test was conducted and the device used. A “y” in the stasis column indicates successful embolization of the vessel as confirmed with angiography at the time of endoluminal cauterization.

TABLE 2

| | Vessel | Device | Stasis |
|---|---|---|---|
| SW001 | L Ext Iliac | Basket | Y |
| | R Ext Iliac | Basket | Y |
| | Aorta | Basket | Y |
| SW002 | L Ext Iliac | Basket | Y |
| | R Ext Iliac | Basket | N |
| | Aorta | Basket | N |
| | L Int Iliac | Basket | Y |
| | R small lumbar branch | Cope Mandril | Y |
| | SMA gut arcade | Cope Mandril | Y |
| | Mid R. Colic | Interlock | Y |
| SW003 | R external Iliac | Basket | Y |
| | L external Iliac | Basket | Y |
| | L kidney upper pole | Cope Mandril | Y |
| | L kidney lower pole | Cope Mandril | Y |
| | L kidney mid pole to main renal | Cope Mandril | Y |
| | L kidney main renal | Cope Mandril | Y |

Advantageously, techniques have been disclosed herein that facilitate the removal of an implanted device from a subject. In particular, a conductive retrieval apparatus is coupled to a conductive retrieval portion of the implanted device and electrical current is supplied to the implanted device. The current is conveyed through the implanted device to conductive portions of the device in contact with tissue of the subject, thereby burning through any tissue adhered to the implanted device.

What is claimed is:

1. A method of removing an implantable device comprising the steps of:
coupling a retrieval apparatus to a retrieval portion of an implantable device, wherein the implantable device includes a plurality of expandable members each having a portion that comes into contact with a tissue of a subject when expanded;
delivering, via the retrieval apparatus, electrical current to the portions of the expandable members that come into contact with the tissue of the subject; and
providing a force to the retrieval portion of the implantable device to collapse the expandable members.

2. The method of claim 1, wherein the implantable device is an inferior vena cava (IVC) filter, and wherein the portions of the expandable members that come into contact with the tissue comprise anchor members located at a distal end of the filter.

3. The method of claim 1, wherein the implantable device is a stent, and wherein the expandable members form a substantially cylindrical structure when expanded.

4. The method of claim 1, wherein the implantable device is an embolization basket, and wherein the expandable members are coupled at an end opposite the retrieval portion.

5. The method of claim 1, wherein the retrieval apparatus includes a conductive snare, and wherein the retrieval portion of the implantable device includes a conductive hook.

6. The method of claim 1, further comprising controlling an amount of electrical current delivered by the retrieval apparatus to the portion of the portions of the expandable members that come into contact with the tissue of the subject.

7. The method of claim 1, wherein the current is delivered via the retrieval apparatus for a period of time between 30 seconds and 3.5 minutes.

8. The method of claim 1, wherein the tissue has overgrown at least a portion of a particular expandable member that comes into contact with the tissue and the retrieval apparatus delivers an amount of current sufficient to burn the overgrown tissue and facilitate removal of the implantable device.

9. The method of claim 8, wherein the tissue comprises a tumor.

10. The method of claim 1, further comprising:
inserting the implantable device into the subject; and
expanding the implantable device.

11. The method of claim 1, wherein the portions of the expandable members that come into contact with the tissue comprise hooks.

12. The method of claim 1, further comprising:
removing the implantable device from the subject.

13. An implantable device comprising:
a plurality of expandable members each having a portion that comes into contact with a tissue of a subject when expanded during implantation of the device into the subject; and
a retrieval portion coupled to the plurality of elongated members configured to collapse the expandable members in response to an applied force,
wherein the retrieval portion and the portions of the expandable members that come into contact with the tissue comprise conductive material and are electrically coupled.

14. The device of claim 13, wherein the implantable device is an inferior vena cava (IVC) filter, and wherein the portions of the expandable members that come into contact with the tissue comprise anchor members located at a distal end of the filter.

15. The device of claim 13, wherein the implantable device is a stent, and wherein the expandable members form a substantially cylindrical structure when expanded.

16. The device of claim 13, wherein the implantable device is an embolization basket, and wherein the expandable members are coupled at an end opposite the retrieval portion.

17. The device of claim 13, wherein the retrieval portion comprises a hook.

18. A retrieval apparatus for an implantable device comprising:
an electrical power supply;
a current regulator coupled to the power supply that regulates electrical current from the power supply; and
a conductive snare coupled to the power supply and current regulator configured to provide a retrieval force and the electrical current from the power supply to a retrieval portion of an implantable device.

19. The retrieval apparatus of claim 18, wherein the power supply is configured to provide between 0.2 and 0.55 amperes to the conductive snare.

20. The retrieval apparatus of claim 18, wherein the current is delivered via the retrieval apparatus for a period of time between 30 seconds and 3.5 minutes.
